(19) United States
(12) Patent Application Publication
(10) Pub. No.: US 2007/0130631 A1
(43) Pub. Date: Jun. 7, 2007

(54) TAAR1 transgenic animal

(76) Inventors: Marius Hoener, Basel (CH); Lothar Lindemann, Basel (CH); Claas Aiko Meyer, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/602,010

(22) Filed: Nov. 20, 2006

(30) Foreign Application Priority Data
Dec. 5, 2005 (EP) 05111699.4

(51) Int. Cl. A01K 67/027; C12N 5/06

(52) U.S. Cl. 800/18; 435/320.1; 435/354

(57) ABSTRACT

The present invention relates to genetic constructs comprising DNA encoding TAAR1 and their use to generate animals transgenic for TAAR1, transgenic animals comprising in their genome transgenic DNA encoding TAAR1, their use and methods of generating these animals.

### PRIORITY TO RELATED APPLICATIONS

This application claims the benefit of European Patent Application No. 05111699.4, filed Dec. 2, 2005. The entire contents of the above-identified application is hereby incorporated by reference.

### FIELD OF THE INVENTION

The present invention generally provides genetic constructs comprising a DNA sequence encoding TAAR1 operatively linked to a promoter.

### SUMMARY OF THE INVENTION

The present invention provides genetic constructs comprising DNA encoding TAAR1 and methods for producing non-human transgenic animals comprising within their genome transgenic DNA encoding TAAR1. Furthermore, the present invention provides non-human transgenic animals comprising within their genomes transgenic DNA encoding TAAR1. Moreover, transgenic animals overexpressing TAAR1, as well as the methods of producing them are also provided. The invention also relates to the use of these animals as a tool for assessing TAAR1 function, for further characterization of known TAAR1 ligands and for identifying as yet unknown ligands of TAAR1, for analyzing TAAR1 signal transduction mechanisms, for analyzing the physiological function of TAAR1 in vivo, and for characterizing agonists, antagonists or modulators of TAAR1. Furthermore, the invention relates to the use of the said TAAR1 transgenic animals for studying the function of TAAR1 and their use as a model for identifying and testing the therapeutic effect of compounds regarding neurological, psychiatric and metabolic disorders.

The present invention therefore provides genetic constructs comprising a DNA sequence encoding TAAR1 operatively linked to a promoter.

The present invention further provides a method of producing a transgenic non-human animal whose genome comprise transgenic DNA encoding TAAR1, comprising
a) introducing a genetic construct as described above into a non-human zygote or an non-human embryonic stem cell,
b) generating a transgenic non-human animal from said zygote or embryonic stem cell, and thereby,
c) producing a transgenic non-human animal whose genome comprises transgenic DNA encoding TAAR1.

A further embodiment of the invention provides a method of producing a non-human transgenic animal expressing transgenic TAAR1 comprising
a) introducing a genetic construct as described above into a non-human zygote or an non-human embryonic stem cell,
b) generating a transgenic non-human animal from said zygote or embryonic stem cell, and thereby,
c) producing a transgenic non-human animal expressing transgenic TAAR1.

### BACKGROUND OF THE INVENTION

Trace amines (TAs) are endogenous compounds related to biogenic amine neurotransmitters which are present in the mammalian nervous system in trace amounts. The intense research efforts on the pharmacology and metabolism of trace amines during the last decades has been triggered by their tight link to a variety of highly prevalent conditions such as depression, schizophrenia, anxiety disorders, bipolar disorder, attention deficit hyperactivity disorder, neurological diseases such as Parkinson Disease, epilepsy, migraine, hypertension, substance abuse and metabolic disorders such as eating disorders, diabetes, obesity and dyslipidemia (Reviewed in: Lindemann & Höner, 2005; Branchek, T. A. and Blackburn, T. P. (2003) Curr. Opin. Pharmacol. 3, 90-97; Premont, R. T., Gainetdinov, R. R., Caron, M. G. (2001) Proc. Natl. Acad. Sci. 98, 9474-9475.; Davenport, A. P. (2003) Curr. Opin. Pharmacol. 3,127-134.). However, a detailed understanding of the physiology of trace amines at the molecular level has only become possible with the recent identification of a novel family of G protein-coupled receptors termed Trace Amine Associated Receptors (TAARs; Lindemann, L., Ebeling, M., Kratochwil, N. A., Bunzow J. R., Grandy, D. K., Hoener, M. C. (2005) Genomics 85, 372-385; Bunzow, et al. (2001) Mol. Pharmacol. 60, 1181-1188; Borowsky, B., et al., (2001). Proc. Natl. Acad. Sci. U. S. A. 98, 8966-8971). Some of these receptors display sensitivity to trace amines, and their unique pharmacology and expression pattern make these receptors prime candidates for targets in drug development in the context of several diseases, some of which previously had been linked to trace amines. Progress in understanding the physiological relevance of TAARs and their ligands on the systems level critically depends on a detailed knowledge of their expression pattern, their pharmacology and the modes of signal transduction. In this context, compounds acting as agonists, antagonists or positive or negative modulators on TAARs, as well as transgenic animal models overexpressing the TAAR1 receptor are essential tools for dissecting the molecular function of this receptor family and to fully understand their potential relevance as targets in drug development.

### BRIEF DESCRIPTION OF THE FIGURES

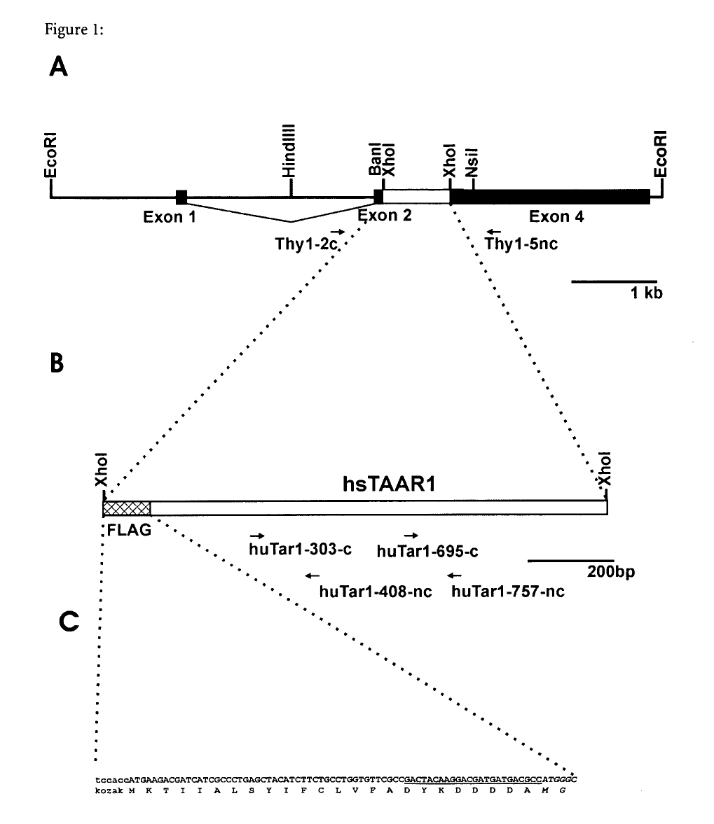

FIG. 1 shows a schematic representation of the genetic construct huTAAR-TG incorporated in the plasmid pThy1-hsTAAR1 (not shown). The control region of the Thy1 gene was used to control the expression of a human TAAR1 cDNA in the brain.
A: The 6.8 kb control region of the Thy1 gene in which a 1.5 kbp BanI-XhoI fragment located on exons 2 and 4 was replaced by a XhoI cloning site thereby deleting Exon 3. The present exons (Exon 1, Exon 2, Exon 4) of the Thy1 gene are indicated in black.
B: The XhoI site in the Thy1 gene was used to insert an XhoI fragment containing the human TAAR1 cDNA (hsTAAR1, white). The human TAAR1 cDNA was fused with a N-terminal tag (FLAG, hatched).
C: The N-terminal tag is shown as nucleotide sequence and as its translation into single letter amino acid code. The Kozak sequence (small letters) is followed by a signal sequence of the influenza hemaglutinin virus (capital letters), the FLAG epitope tag (capital letters, underlined) and the Met-Gly linker (capital letters, italic).

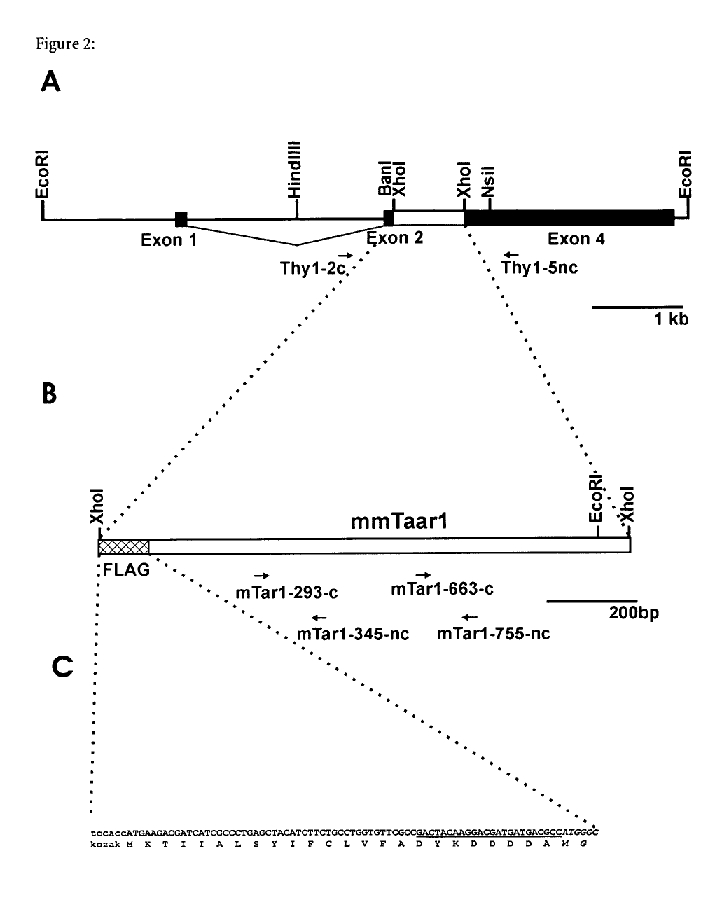

FIG. 2: shows a schematic representation of the mmTAAR-TG construct incorporated in the plasmid pThy1-mmTaar1 (not shown). The control region of the Thy1 gene was used to control the expression of a human Taar1 cDNA in the brain.
A: The 6.8 kb control region of the Thy1 gene in which a 1.5 kbp BanI-XhoI fragment located on exons 2 and 4 was replaced by a XhoI cloning site thereby deleting Exon 3. The present exons (Exon 1, Exon 2, Exon 4) of the Thy1 gene are indicated in black.
B: The XhoI site in the Thy1 gene was used to insert an XhoI fragment containing the human Taar1 cDNA (hsTaar1, white). The human Taar1 cDNA was fused with a N-terminal tag (FLAG, hatched).
C: The N-terminal tag is shown as nucleotide sequence and as its translation into single letter amino acid code. The Kozak sequence (small letters) is followed by a signal sequence of the influenza hemaglutinin virus (capital letters), the FLAG epitope tag (capital letters, underlined) and the Met-Gly linker (capital letters, italic).

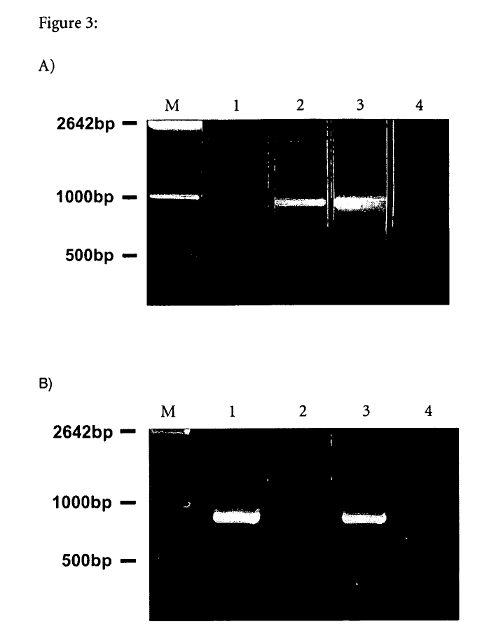

FIG. 3: shows the genotyping of transgenic lines and B6-Tg(TAAR1)1 and B6-Tg(Taar1)2 by means of PCR. DNA was analyzed for the presence of the transgene, indicating the respective genotypes of the animals. Marker XIV (Roche) was used as molecular weight standard.
A: B6-Tg(Taar1)2: Ethidium bromide stained agarose gel of PCR reaction to detect the presence of the Thy1-mmTaar1 sequences in tail biopsies of mice.
M: Marker XIV, 1: mouse biopsy 9; 2: mouse biopsy 10; 3: positive control; 4: negative control.
B: B6-Tg(TAAR1) 1: Ethidium bromide stained agarose gel of PCR reaction to detect the presence of the Thy1-hsTaar1 sequences in tail biopsies of mice.
M: Marker XIV, 1: mouse biopsy 34; 2: mouse biopsy 35; 3: positive control; 4: negative control.

### DETAILED DESCRIPTION OF THE INVENTION

Definitions

The term “promoter” as used herein refers to DNA regions which control the expression of a coding sequence.

The term “transgenic DNA” as used herein describes DNA artificially introduced and incorporated into an organism.

The term “sequence of taar1 gene” as used herein, describes the DNA sequence encoding TAAR1.

The term “transgenic TAAR1” as used herein describes TAAR1 protein originating from DNA artificially introduced and incorporated into an organism.

The term “transgenic animal” as used herein describes an animal comprising transgenic DNA in their genome. This transgenic DNA may be incorporated somewhere in the genome.

All publications, patents and references cited herein are hereby incorporated by reference in their entirety.

### DETAILED DESCRIPTION

The present invention provides genetic constructs comprising DNA encoding TAAR1 and methods for producing non-human transgenic animals comprising within their genome transgenic DNA encoding TAAR1. Furthermore, the present invention provides non-human transgenic animals comprising within their genomes transgenic DNA encoding TAAR1. Moreover, transgenic animals overexpressing TAAR1, as well as the methods of producing them are also provided. The invention also relates to the use of these animals as a tool for assessing TAAR1 function, for further characterization of known TAAR1 ligands and for identifying as yet unknown ligands of TAAR1, for analyzing TAAR1 signal transduction mechanisms, for analyzing the physiological function of TAAR1 in vivo, and for characterizing agonists, antagonists or modulators of TAAR1. Furthermore, the invention relates to the use of the said TAAR1 transgenic animals for studying the function of TAAR1 and their use as a model for identifying and testing the therapeutic effect of compounds regarding neurological, psychiatric and metabolic disorders.

The sequence of TAAR1 gene can be derived from any animal. Preferably the sequence of taar1 derives from a mammal, more preferably, the sequence of taar1 is a human (NM_{—}138327, SEQ. ID NO:1)or a mouse sequence (NM_{—}053205, SEQ. ID NO: 2).

Preferably, the present invention provides a genetic construct comprising a DNA sequence encoding TAAR1 operatively linked to a promoter, wherein the coding sequence is a human sequence and the promoter is selected from the group consisting of a tissue-specific promoter, a brain-specific promoter and a controllable promoter.

The promoter may be a neuronal promoter. In a preferred embodiment the promoter is a tissue-specific promoter. A tissue-specific promoter may be any promoter, which controls and directs expression of a gene in a tissue-specific manner, e.g., in brain tissue (brain-specific), in muscle tissue, in liver tissue, in kidney tissue, etc. Preferably, the promoter provides specific expression in the central nervous system (CNS). Most preferably, the promoter is the mouse Thy1 promoter (Ingraham, H. A., Lawless, G. M., Evans, G. A. (1986) The mouse Thy-1.2 glycoprotein gene: complete sequence and identification of an unusual promoter. J Immunol. 136, 1482-1489).

The promoter may also be a controllable promoter. A controllable promoter may be any promoter, which controls the expression of a transgene in a regulatable and/or inducible fashion, e.g., by addition of specific inducer or repressor substances. Several inducible bacterial promoter are known in the art (Schultze N, Burki Y, Lang Y, Certa U, Bluethmann H; Nat Biotechnol 1996; 14(4): 499-503; van der Neut R; Targeted gene disruption: applications in neurobiology; J Neurosci Methods 1997; 71(1): 19-27; Liu H S, Lee C H, Lee C F, Su I J, Chang T Y; Lac/Tet dual-inducible system functions in mammalian cell lines. Biotechniques. 1998; 24(4): 624-8, 630-2).

The genetic construct may additionally comprises a Kozak Sequence. Preferably, the genetic construct comprises an optimized Kozak sequence such as for example the sequence: tccacc. The Kozak Sequence is a DNA sequence that surrounds the ATG start signal for the translation of an mRNA (Kozak, M., An analysis of 5′-noncoding sequences from 699 vertebrate messenger RNAs. Nucleic Acids Res. 15 (1989) 8125-8148.).

In a preferred embodiment, the genetic construct comprise additionally an N-terminal influenza hemaglutinin viral leader sequence. In another preferred embodiment the genetic construct comprises additionally an epitope marker such as for example M1-FLAG epitope. In another preferred embodiment, the genetic construct comprise additionally a Met-Gly linker. In a even more preferred embodiment the genetic construct comprises an N-terminal influenza hemaglutinin viral leader sequence, an M1-FLAG epitope and a Met-Gly linker (Bunzow, et al. (2001) Mol. Pharmacol. 60,1181-1188; X. M. Guan, T. S. Kobilka, B. K. Kobilka (1992) Enhancement of membrane insertion and function in a type IIIb membrane protein following introduction of a cleavable signal peptide. J. Biol. Chem. 267, 21995-21998).

Preferably, the genetic construct is as depicted in FIG. 1 or FIG. 2.

More preferably, the genetic construct is Thy1-hsTAAR1 incorporated in the plasmid pThy1-hsTAAR1 deposited under accession number DSM 17761 (deposited under the Budapest Treaty at the Deutsche Sammlung von Microorganismen und Zellkulturen GmbH (DSMZ) with an effective deposition date of 30.11.2005), or the genetic construct is Thy1-mmTAAR1 incorporated in the plasmid pThy1-mmTaar1 deposited under accession number DSM 17760 (deposited under the Budapest Treaty at the Deutsche Sammlung von Microorganismen und Zellkulturen GmbH (DSMZ) with an effective deposition date of 30, Nov. 2005).

The present invention further provides a method of producing a transgenic non-human animal whose genome comprise transgenic DNA encoding TAAR1, comprising
a) introducing a genetic construct as described above into a non-human zygote or an non-human embryonic stem cell,
b) generating a transgenic non-human animal from said zygote or embryonic stem cell, and thereby,
c) producing a transgenic non-human animal whose genome comprises transgenic DNA encoding TAAR1.

A further embodiment of the invention provides a method of producing a non-human transgenic animal expressing transgenic TAAR1 comprising
d) introducing a genetic construct as described above into a non-human zygote or an non-human embryonic stem cell,
e) generating a transgenic non-human animal from said zygote or embryonic stem cell, and thereby,
f) producing a transgenic non-human animal expressing transgenic TAAR1.

The zygote or embryonic stem cell may derive from any non-human animal. Preferably, the zygote or embryonic stem cell derives from a rodent. More preferably, the zygote or embryonic stem cell derives from a mouse.

Preferably, the zygote used in the methods described above is a C57BL/6J zygote. Zygotes used in the art which may also be used in the methods of this invention comprise, but are not limited to, CBA/zygotes, BALB/c zygotes, DBA/2 zygotes and SV129 zygotes.

Embryonic stem cells used in the art which may also be used in the methods of this invention comprise but are not limited to embryonic stem cells derived from mouse strains such as C57BL/6, CBA/, BALB/c, DBA/2 and SV129. Preferably, embryonic stem cells derived from C57BL/6 mice are used (Seong, E et al (2004) Trends Genet. 20, 59-62; Wolfer, D. P. et al., Trends Neurosci. 25 (2002): 336-340).

The present invention further provides the transgenic non-human animal produced by any of the above described methods.

For example, the transgenic animals may be generated by injecting above described DNA construct into the pronucleus of zygotes, transferring these injected zygotes into pseudo-pregnant foster mothers, breeding founder animals resulting from the oocytes to wild type animals, testing the offspring resulting from these breedings for the presence of the synthetic DNA transgene construct, breeding hemizygous animals, optionally to generate homozygous transgenic animals.

Alternatively, the transgenic animals may be generated by introducing the genetic construct as described above into embryonic stem cells and subsequently selecting embryonic stem cell clones for the presence of the transgene in the genome, verifying the presence of the transgene in the transformed embryonic stem cell clones, injecting the verified recombinant embryonic stem cells into blastocysts of wild type animals, transferring these injected blastocysts into pseudo-pregnant foster mothers, breeding chimeras resulting from the blastocysts to wild type animals, testing the offspring resulting from these breedings for the presence of the transgene, breeding hemizygous animals, optionally to generate homozygous transgenic animals.

In one embodiment of the invention, transgenic non-human animals whose genome comprises transgenic DNA encoding TAAR1 are provided. In a preferred embodiment the transgenic non-human animal comprises a genetic construct as depicted in FIG. 1 or a genetic construct as depicted in FIG. 2.

In another preferred embodiment, the transgenic non-human animal comprises a genetic construct Thy1-hsTAAR1 incorporated in the plasmid pThy1-hsTAAR1 deposited under accession number DSM 17761 (deposited under the Budapest Treaty at the Deutsche Sammlung von Microorganismen und Zellkulturen GmbH (DSMZ) with an effective deposition date of 30, Nov. 2005), or the genetic construct is Thy1-mmTaaR1 incorporated in the plasmid pThy1-mmTaar1 deposited under accession number DSM 17760 (deposited under the Budapest Treaty at the Deutsche Sammlung von Microorganismen und Zellkulturen GmbH (DSMZ) with an effective deposition date of 30, Nov. 2005).

In another embodiment transgenic non-human animals expressing transgenic TAAR1 are provided. In a preferred embodiment, the transgenic TAAR1 is expressed in a tissue-specific manner, i.e., in the brain. In another embodiment, the expression of the transgenic TAAR1 may be temporally controlled, e.g., by addition of specific inducer or repressor substances. In a preferred embodiment, the transgenic non-human animal overexpresses the transgenic TAAR1 protein. In a more preferred embodiment, the transgenic TAAR1 is overexpressed in tissue-specific manner.

The transgenic non-human animal may be any non-human animal known in the art, which may be used for the methods of the invention. Preferably, the transgenic non-human animal is a mammal, more preferably the transgenic non-human animal is a rodent. Most preferably, the transgenic animal of the invention is a mouse.

The transgenic non-human animals described above may be analyzed genetically, molecularly and behaviorally.

The present invention also relates to descendants of the transgenic non-human animals as provided by the invention, obtained by breeding with the same or with another genotype. A descendant is used herein as a synonym for progeny.

The transgenic animals may be used for preparing primary cell cultures, and for the preparation of secondary cell lines derived from primary cell preparations of these animals or their descendants. Furthermore, the transgenic animals can be used for the preparation of tissue or organotypic brain slice cultures. Moreover, the transgenic animals can be used for the preparation of tissue explants or organ explants or cultures thereof. In addition, the transgenic animal may be used for the preparation of tissue or cell extracts such as membrane or synaptosomal preparations.

The present invention further provides a cell line or primary cell culture derived from the transgenic non-human animals as provided by the invention or their descendants.

Furthermore, the present invention provides a tissue or organotypic brain slice culture, derived from the transgenic non-human animals as provided by the invention or their descendants.

In addition, the present invention provides tissue explants or organ explants or cultures thereof, derived from the transgenic non-human animals as provided by the invention or their descendants.

Moreover, the present invention provides a tissue extract or a cell extract, derived from the transgenic non-human animals as provided by the invention or their descendants.

Cell culture based models can be prepared by two methods. Cell cultures can be isolated from the non-human transgenic animals or prepared from established cell cultures using the same genetic constructs as described above with standard cell transfection techniques.

Integration of the genetic construct comprising transgenic DNA encoding TAAR1, can be detected by various methods comprising genomic Southern blot and PCR analysis using DNA isolated from tissue biopsies of the animals.

It will be apparent to the person skilled in the art that there are a large number of analytical procedures which may be used to detect the expression of the transgenic DNA comprising methods at the RNA level comprising mRNA quantification by reverse transcriptase polymerase chain reaction (RT-PCR) or by Northern blot, in situ hybridization, as well as methods at the protein level comprising histochemistry, immunoblot analysis and binding studies. Quantification of the expression levels of the transgene can moreover be determined by the ELISA technology, which is common to those knowledgeable in the art.

Quantitative measurement can be accomplished using many standard assays. For example, transcript levels can be measured using RT-PCR and hybridization methods including RNase protection, Northern blot analysis, and RNA dot blot analysis. Protein levels can be assayed by ELISA, Western blot analysis, and by comparison of immunohistochemically or histochemically stained tissue sections. Immunohistochemical staining, enzymatic histochemical stainings as well as immuno-electron microscopy can also be used to assess the tissue distribution of the endogenous as well as of the overexpressed TAAR1 protein.

The transgenic animals of the invention may be further characterized by methods known in the art, comprising immunohistochemistry, electron microscopy, Magnetic Resonance Imaging (MRI), Positron Emission Tomography (PET) and by behavioral and physiological studies addressing neurological, sensory, and cognitive functions as well as physiological (e.g. metabolic) parameters. Examples of behavioral tests and physiological examinations are: Spontaneous behavior, behavior related to cognitive functions, pharmacologically-disrupted behavior, grip strength test, horizontal wire test, forced swim test, rotarod test, locomotor activity test, prepulse inhibition test, Morris water maze test, Y-maze test, light-dark preference test, passive and active avoidance tests, marble burying test, plus maze test, learned helplessness test, stress-induced hyperthermia, measuring food consumption and development of body weight over time, measuring body temperature and energy consumption under resting and basal conditions and during heat and cold exposure, determining the thermoneutral zone, determining the food assimilation coefficient (e.g. by bomb calorimetry), determining the energy assimilation and the energy content of feces, determining the respiratory coefficient e.g. for analysis of the carbohydrate and lipid metabolism, determining the substrate utilization and energy expenditure during food restriction, determining the oxygen consumption, CO₂— and heat production e.g. by indirect calorimetry, measuring the heart rate and blood pressure under resting, basal and stress conditions (e.g. by telemetry), determining the body composition (e.g. regarding water content, fat amount and fat-free mass).

A further objective of the present invention is the use of a non-human transgenic animal, a cell line or primary cell culture, a tissue or organotypic brain slice cultures, a tissue explant or an organ explant or culture thereof, a tissue or cell extract derived from the transgenic non-human animals as provided by the invention or its descendants as a model for identifying and testing the therapeutic effect of a compound in disorders comprising depression, anxiety disorders, bipolar disorder, attention deficit hyperactivity disorder, stress-related disorders such as posttraumatic stress disorder, psychotic disorders such as schizophrenia, neurological diseases such as Parkinson's Disease, neurodegenerative disorders such as Alzheimer's Disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

Moreover, the present invention provides the use of a non-human transgenic animal, a cell line or primary cell culture, a tissue or organotypic brain slice cultures, a tissue explant or an organ explant or culture thereof, a tissue or cell extract derived from the transgenic non-human animals as provided by the invention or its descendants as a model for studying the susceptibility to compounds inducing disorders comprising depression, anxiety disorders, bipolar disorder, attention deficit hyperactivity disorder, stress-related disorders such as posttraumatic stress disorder, psychotic disorders such as schizophrenia, neurological diseases such as Parkinson's Disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

A further objective of the present invention is the use of a non-human transgenic animal, a cell line or primary cell culture, a tissue or organotypic brain slice cultures, a tissue explant or an organ explant or culture thereof, a tissue or cell extract derived from the transgenic non-human animals as provided by the invention or its descendants as a tool for assessing TAAR1 function.

Furthermore, the present invention provides the use of a non-human transgenic animal, a cell line or primary cell culture, a tissue or organotypic brain slice cultures, a tissue explant or an organ explant or culture thereof, a tissue or cell extract derived from the transgenic non-human animals as provided by the invention or its descendants as a tool for identifying unknown ligands of TAAR1 and their characterization.

Moreover, the present invention provides the use of a non-human transgenic animal, a cell line or primary cell culture, a tissue or organotypic brain slice cultures, a tissue explant or an organ explant or culture thereof, a tissue or cell extract derived from the transgenic non-human animals as provided by the invention or its descendants as a tool for determining the specificity of compounds acting on TAAR1.

A further objective of the present invention is the use of a non-human transgenic animal, a cell line or primary cell culture, a tissue or organotypic brain slice cultures, a tissue explant or an organ explant or culture thereof, a tissue or cell extract derived from the transgenic non-human animals as provided by the invention or its descendants for studying the intracellular trafficking of TAARs or of other cellular components linked to TAARs.

The invention further provides genetic constructs, methods, transgenic animals, test systems and uses substantially as described herein before especially with reference to the foregoing examples.

Having now generally described this invention, the same will become better understood by reference to the specific examples, which are included herein for purpose of illustration only and are not intended to be limiting unless otherwise specified, in connection with the following figures.

### EXAMPLES

Commercially available reagents referred to in the examples were used according to manufacturer's instructions unless otherwise indicated.

### Example 1

### Construction of the pThy1-hsTAAR1 and pThy1-mmTaar1 Plasmid Constructs PCR Amplification of the cDNA Sequences

Oligonucleotides were designed based on the published mouse TAAR1 coding sequence (Genbank Accession No. NM_{—}053205, SEQ. ID NO: 2) or human TAAR1 coding sequence (Genbank Accession No. NM_{—}138327, SEQ. ID NO: 1) or on other elements of the p Thy1-hsTAAR1 and pThy1-mmTaar1 plasmids and they were obtained from Microsynth AG (Balgach, Switzerland). All molecular cloning techniques were carried out essentially according to Sambrook et. al. (Molecular Cloning: A laboratory manual. 1989, Cold Spring Harbor Laboratory Press, Cold Spring Harbor.) and the instructions of the suppliers of kits and enzymes.

The coding sequences of human (hsTAAR1) and mouse (mmTaar1) were amplified by PCR from genomic C57BL/6 DNA (mmTaar1) or a cloned coding sequence pcDNA3.1-humTar01 (hsTAAR1).

The 5′-oligonucleotides, hTar1-tg-3c (SEQ. ID NO: 21) and mTar1-tg-1c (SEQ. ID NO: 27), were designed to contain a XhoI site, a Kozak sequence, sequences coding for a signal sequence of the influenza hemaglutinin virus, the FLAG epitope Tag and a Met-Gly linker (FIG. 1C, FIG. 2C). The 3′-oligonucleotides, hTar1-tg-4nc (SEQ. ID NO: 22) and mTar1-tg-2nc (SEQ. ID NO: 28) introduce a XhoI site immediately downstream of the stop codon.

hsTAAR1-PCR Amplification

0.5 ng/ul template DNA (pcDNA3.1-humTar01), 300 μM dNTPs (PCR Nucleotide Mix, Roche Diagnostics, Mannheim, Germany), 1 μM of oligonucleotide huTar1-tg-3c, 1 μM of oligonucleotide huTar1-tg-4nc 1× PCR buffer 3 (part of the Expand High Fidelity PCR System; Roche Diagnostics, Mannheim, Germany), 0.075 U/μl Expand Polymerase Mix (part of the Expand High Fidelity PCR System; Roche Diagnostics, Mannheim, Germany)) in a total volume of 20 μl were incubated with the following protocol: 94° C. 2 min., 10×(94° C. 10 sec., 60° C. 30 sec., 68° C. 2 min.), 20×(94° C. 10 sec., 60° C. 30 sec., 68° C. 2 min.+15 sec/cycle) 68° C. 7 min, ∞ 12° C. on a PCR Thermocycler MJ Research PTC-200 (MJ Research Inc., Watertown, USA).

mmTaar1-PCR Amplification

0.5 ng/μl template DNA (genomic C57BL/6 DNA), 300 μM dNTPs (PCR Nucleotide Mix, Roche Diagnostics, Mannheim, Germany), 1 μM of oligonucleotide mTar1-tg-1c, 1 μM of oligonucleotide mTar1-tg-2nc, 1× PCR buffer 3 (part of the Expand High Fidelity PCR System; Roche Diagnostics, Mannheim, Germany), 0.075 U/μl Expand Polymerase Mix (part of the Expand High Fidelity PCR System; Roche Diagnostics, Mannheim, Germany)) in a total volume of 20 μl were incubated with the following protocol: 94° C. 2 min., 10×(94° C. 10 sec., 60° C. 30 sec., 68° C. 2 min.), 20×(94° C. 10 sec., 60° C. 30 sec., 68° C. 2 min.+15 sec/cycle) 68° C. 7 min, ∞ 12° C. on a PCR Thermocycler MJ Research PTC-200 (MJ Research Inc., Watertown, USA).

Cloning into the Cloning Vector pCMV

The resulting 1.1 kb PCR amplification products were separated using agarose gel-electrophoresis followed by purification with the StrataPrep PCR Purification Kit (Stratagene, La Jolla, Calif., USA). The purified PCR products were then subcloned into the SrfI site of the pCMVScript vector of the pCMV PCR Cloning Kit (Invitrogen-Gibco, Carlsbad, Calif., USA) according to the kits instructions. The resulting vector are pCMV-mmTaar1 and pCMV-hsTAAR1.

Orientation and sequence were confirmed by sequencing using the BigDye Terminator v1.1 Cycle Sequencing Kit (Applied Biosystems) and an ABIPrism 310 Genetic Analyzer with the oligonucleotides depicted in table 1:

TABLE 1
Sequencing reactions of pCMV-mmTaar1 and pCMV-hsTAAR1

| | Oligonucleotides used | | |
|---|---|---|---|
| | for sequencing | | Resulting |
| Vector | Term | SEQ. ID NO: | sequence: |
| pCMV-hsTAAR1 | T3 forward | 29 | SEQ. ID NO: 5 |
| pCMV-hsTAAR1 | T7 reverse | 30 | SEQ. ID NO: 6 |
| pCMV-hsTAAR1 | hu-Tar 303c | 17 | SEQ. ID NO: 7 |
| pCMV-hsTAAR1 | hu-Tar 408nc | 18 | SEQ. ID NO: 8 |
| pCMV-hsTAAR1 | hu-Tar 695 c | 19 | SEQ. ID NO: 9 |
| pCMV-hsTAAR1 | hu-Tar 757 nc | 20 | SEQ. ID NO: 10 |
| pCMV-mmTaar1 | T3 forward | 29 | SEQ. ID NO: 11 |
| pCMV-mmTaar1 | T7 reverse | 30 | SEQ. ID NO: 12 |
| pCMV-mmTaar1 | mTar-293c | 23 | SEQ. ID NO: 13 |
| pCMV-mmTaar1 | mTar-345nc | 24 | SEQ. ID NO: 14 |
| pCMV-mmTaar1 | mTar-663c | 25 | SEQ. ID NO: 15 |
| pCMV-mmTaar1 | mTar-755nc | 26 | SEQ. ID NO: 16 |

The sequences of SEQ. ID. NOs: 5-10 were edited manually and the vector sequence of pCMV was omitted. They can be assembled to confirm the correct sequence of the pCMV-hsTAAR1 vector (SEQ. ID NO: 3).

The sequences of SEQ. ID. NOs: 11-16 were edited manually and the vector sequence of pCMV was omitted. They can be assembled to confirm the correct sequence of the pCMV-mmTaar1 vector (SEQ. ID NO: 4).

Cloning into the pThy Vector

The vector pCMV-mmTaar1 and pCMV-hsTAAR1 were used to generate the vectors pThy1-mmTaar1 and pThy1-hsTAAR1, respectively. To this end a pCMV-mmTaar1 and pCMV-hsTAAR1 were digested with XhoI and the resulting 1.1 kb fragment containing the Taar1 coding sequences were purified from agarose gels. They were subsequently ligated into the vector pThy1 (FIG. 1A; FIG. 2A; Richards, J. G., Higgins, G. A., Ouagazzal, A. M., Ozmen, L., Kew, J. N., Bohrmann, B., Malherbe, P., Brockhaus, M., Loetscher, H., Czech, C., Huber, G., Bluethmann, H., Jacobsen, H. and Kemp, J. A. (2003) J. Neurosci. 23(26), 8989-9003)) that has been opened with the restriction enzyme XhoI.

The resulting vectors pThy1-mmTaar1 and pThy1-hsTAAR1 were confirmed using sequencing with Thy1-2c (SEQ. ID NO: 31) and restriction digests.

Deposition data: The plasmid comprising the genetic construct pThy1-mmTaar1 was deposited under the Budapest Treaty at the Deutsche Sammlung von Microorganismen und Zellkulturen GmbH (DSMZ), Mascheroder Weg 1 b, D-38124 Braunschweig, Germany, with an effective deposition date of 30, Nov. 2005 under the accession number DSM 17760.

The plasmid comprising the genetic construct pThy1-hsTAAR1 was deposited under the Budapest Treaty at the Deutsche Sammlung von Microorganismen und Zellkulturen GmbH (DSMZ), Mascheroder Weg 1 b, D-38124 Braunschweig, Germany, with an effective deposition date of 30, Nov. 2005 under the accession number DSM 17761.

### Example 2

Generating transgenic mouse lines B6-Tg(TAAR1)1 and B6-Tg(Taar1)2

All handling of animals was carried out in compliance with Swiss Federal and Cantonal laws on animal research, and permission for the generation and handling of the mutant mouse line was specifically granted from the Kantonale Veterinaramt of Basel City with the Tierversuchgenehmigung No. 192.

The plasmids pThy1-mmTaar1 and pThy1-hsTAAR1 were digested with NotI and PvuI, followed by gel purification of the 7,6 kb fragment containing the Thy1 promotor and Taar1 sequences. These fragments of pThy1-mmTaar1 and pThy1-hsTAAR1 were injected into the pronucleus of fertilized C57BL/6 oocytes which were subsequently transferred into the oviducts of pseudopregnant foster mothers essentially as described in (Gene Targeting. 1999, Second Edition, The Practical Approach Series, Oxford University Press, New York) and Hogan, B., Beddington, R., Costantini, F., und Lacy, E. (Manipulating the mouse embryo. 1994, Second Edition, Cold Spring Harbor Press, Cold Spring Harbor). Offspring delivered by the foster mothers were considered as founder animals and analyzed for the presence of the respective transgenes in the genome.

To this end, genomic DNA was isolated from tail biopsies of adult animals with a MagNAPure LC system for nucleic acid purification (Roche Applied Science, Rotkreuz, Switzerland) according to the instructions of the manufacturer and analyzed for the presence of the TAAR1 transgenes by means of PCR.

Transgenic animals comprising in their genome the genetic construct “Thy1-hsTAAR1” are designated as B6-Tg(TAAR 1) 1 and transgenic animals comprising in their genome the genetic construct “Thy1-mmTaar1” are designated as B6-Tg(Taar1)2.

Genotyping of B6-Tg(TAAR1)1

0.5 ng/μl tail biopsy DNA, 200 μM dNTPs (PCR Nucleotide Mix, Roche Diagnostics, Mannheim, Germany), 1 μM of oligonucleotide Thy1-2c (SEQ. ID. NO: 31), 1 μM of oligonucleotide huTar1-757-n (SEQ. ID NO:20)c, 1× PCR reaction buffer (Roche Diagnostics, Mannheim, Germany), 0.05 U/μl Taq DNA Polymerase (Roche Diagnostics, Mannheim, Germany)) in a total volume of 20 μl were incubated with thefollowingprotocol: 94° C. 2 min., 30×(94° C. 20 sec., 60° C. 30 sec., 72° C. 50 sec) 72° C. 7 min, ∞ 12° C. on a PCR Thermocycler MJ Research PTC-200 (MJ Research Inc., Watertown, USA).

Genotyping of B6-Tg(Taar1)2

0.5 ng/μl tail biopsy DNA, 200 μM dNTPs (PCR Nucleotide Mix, Roche Diagnostics, Mannheim, Germany), 1 μM of oligonucleotide mTar1-293-c, 1 μM of oligonucleotide Thy1-5-nc (SEQ. ID NO: 32), 1× PCR reaction buffer (Roche Diagnostics, Mannheim, Germany), 0.05 U/μl Taq DNA Polymerase (Roche Diagnostics, Mannheim, Germany)) in a total volume of 20 μl were incubated with the following protocol: 94° C. 2 min., 30×(94° C. 20 sec., 64° C. sec., 72° C. 50 sec) 72° C. 7 min, ∞ 12° C. on a PCR Thermocycler MJ Research PTC-200 (MJ Research Inc., Watertown, USA).

The PCR products were analyzed by standard agarose gel electrophoresis as described in Sambrook et al. (1989). See FIG. 3 for an example of genotyping.

Three founders were identified with an insertion of the human TAAR1 construct and designated as B6-Tg(TAAR1)1.6, B6-Tg(TAAR1)1.7 and B6-Tg(TAAR1)1.29.

Three founders were identified with an insertion of the murine Taar1 construct and designated as B6-Tg(Taar1)2.15, B6-Tg(Taar1)2.19 and B6-Tg(Taar1)2.27.

Semiquantitative Analysis of Human TAAR1 and Mouse TAAR1 Expression, Respectively, in Brain Regions of the Transgenic Mouse Lines B6-Tg(TAAR1) 1 and B6-Tg(Taar1)2

Offspring of the identified founders B6-Tg (TAAR1)1.6, B6-Tg(TAAR1)1.7 and B6-Tg(TAAR1)1.29 were tested for expression of mRNA coded by the constructs Thy1-hsTAAR1. Offspring of the identified founders B6-Tg(Taar1)2.15, B6-Tg(Taar1)2.19 and B6-Tg(Taar1)2.27 were tested for expression of mRNA coded by the constructs Thy1-mm(Taar1).

To test for expression of the constructs Thy1-mm(Taar1) and Thy1-hsTAAR1 a real time PCR assay was used. To this end total RNA of mouse brains was isolated, transcribed into cDNA and detected with Real-Time PCR using a combination of primers and probe specific for the synthetic N-terminal Tag common to both constructs (FIG. 1C, FIG. 2C).

RNA was prepared from tissue samples essentially according to Chomczynski and Sacchi (Single-step method of RNA isolation by acid guanidinium thiocyanate-phenol-chloroform extraction, Anal. Biochem. 162 (1987) 156-159.). In brief, mice 6 to 8 weeks of age were sacrificed, and total brains were removed and homogenized in 10× volumes of Trizol Reagent (Invitrogen, Paisley, UK) in Lysing Matrix D tubes using a Fast Prep FP120 tissue homogenizer (Savant Instruments, Inc., Holbrook, N.Y.) at a setting of 6 for 20 s. The homogenates were extracted with ⅕ volume of Chloroform and subsequently precipitated using 2 volumes of isopropanol. The precipitated total RNA was washed with 80% Ethanol and dissolved in DEPC-H₂O. 100 μg Total RNA was further purified using the RNeasy Mini Protocol for RNA Cleanup of the RNeasy Mini Kit (Qiagen, Hilden, Germany) including a on-column DNase Digestion with the RNase-Free DNase Set (Qiagen, Hilden, Germany).

For cDNA synthesis the Omniscript Reverse Transkription Kit (Qiagen, Hilden, Germany) was used according to the instructions of the manufacturer. Briefly, 2 μg total RNA were mixed with 1 μM oligo (dT) 15 (Roche Diagnostics, Mannheim, Germany), 0.5 mM of each dNTP (PCR Nucleotide Mix, Roche Diagnostics, Mannheim, Germany), 1× Buffer RT (Qiagen, Hilden, Germany), 10 U RNAse Inhibitor (Roche Diagnostics, Mannheim, Germany) 1 μl Omniscript Reverse Transcriptase (Qiagen, Hilden, Germany) in a total volume of 20 μl. The reaction mix was incubated at 37° C. for 1 h.

Real time PCR was carried out using Custom designed Quantitect Gene expression assays according to the Quantitect Gene Expression Assay Handbook (Qiagen, Hilden, Germany). The transgene specific Quantitect Custom assay was designed to detect the synthetic N-terminal tag and consisted of a forward primer (SEQ. ID NO: 33), reverse Primer (SEQ. ID NO: 34) and FAM labeled probe (SEQ. ID NO: 35). As control the Yakima Yellow labeled QuantiTect Endogenous Control Assay (Mm Arbp 1 YY Quantitect Gene Expression Assay, Qiagen, Hilden, Germany) against the housekeeping gene murine Arbp (Acidic ribosomal phospoprotein PO) was used. 10 μl QuantiTect Probe PCR Master Mix, 2 μl Quantitect Assay Mix and 2 μl cDNA were set up in white reaction plates (MJ Research Inc., Watertown, USA) on a DNA Engine Opticon 2 (MJ Research Inc., Watertown, USA) and real time PCR with the following temperature protocol were performed: 95° C. 15 min., 45×(94° C. 15 sec., 56° C. 30 sec., 76° C. 30 sec). C(T) values were calculated using the Opticon monitor Software (MJ Research Inc., Watertown, USA).

The results of one experiment are summarized in Table 2. In this experiment two mice each of the lines B6-Tg (TAAR1)1.6, B6-Tg(TAAR1)1.7, B6-Tg(TAAR1)1.29, B6-Tg(Taar1)2.15, B6-Tg(Taar1)2.19 and B6-Tg(Taar1)2.27 as well as two wildtype mice were sacrificed, cDNA was produced as described above and two PCR reactions were run for each mouse with the transgene specific Quantitect assay (Qiagen, Hilden, Germany) and, as control, one PCR reaction was run for each mouse with the Arbp specific Quantitect assay (see Table 2).

From wildtype mice, control reactions where the reverse transcriptase was not added, as well as water controls, C(T) values are obtained close or below to the threshold of detection (Table 2). On the other hand, the lines B6-Tg(TAAR1)1.7, B6-Tg(TAAR1)1.29, B6-Tg(Taar1)2.19 and B6-Tg(Taar1)2.27 express the transgenic construct, as C(T) values are within the range of plasmid positive controls (Table 2).

| | Transgene specific | Housekeeping gene |
|---|---|---|
| | probe: | (Arbp) probe: |
| Sample: | C(T) +/− standard | C(T) +/− standard |
| B6-Tg (TAAR1)1.6 | 36.4 +/− 4.2 | 30.7 +/− 0.6 |
| B6-Tg(TAAR1)1.7 | 24.6 +/− 0.8 | 28.5 +/− 2.4 |
| B6-Tg(TAAR1)1.29 | 27.2 +/− 1.6 | 31.6 +/− 2.2 |
| B6-Tg(Taar1)2.15 | 36.2 +/− 3.4 | 29.6 +/− 0.1 |
| B6-Tg(Taar1)2.19 | 23.7 +/− 0.4 | 30.0 +/− 1.8 |
| B6-Tg(Taar1)2.27 | 24.9 +/− 0.6 | 28.0 +/− 1.3 |
| wildtype | 39.6 +/− 3.5 | 28.1 +/− 1.1 |
| Blank (H₂0) | below threshold | nd |
| B6-Tg(Taar1)2.19 without | below threshold | nd |
| reverse transcription | | |
| B6-Tg(TAAR1)1.7 without | below threshold | nd |
| reverse transcription | | |
| pThy1-mmTaar1 (10³ copies) | 33.6 | nd |
| pThy1-mmTaar1 (10⁴ copies) | 29.1 | nd |
| pThy1-mmTaar1 (10⁵ copies) | 25.4 | nd |
| pThy1-mmTaar1 (10⁶ copies) | 22.2 | nd |
| pThy1-mmTaar1 (10⁷ copies) | 19.1 | nd |

Table 2: Results of the real time PCR analysis of cDNAs derived from B6-Tg(TAAR 1)1 and B6-Tg(Taar1)2 mouse brains for the presence of the mRNA transcripts. (C(T): number of PCR cycles; nd: not determined).

1. A genetic construct comprising a DNA sequence encoding TAAR1 operatively linked to a promoter, wherein the coding sequence is a human sequence and the promoter is selected from the group consisting of a tissue-specific promoter, a brain-specific promoter and a controllable promoter.

2. A method of producing a transgenic non-human animal whose genome comprise transgenic DNA encoding TAAR1, comprising
a) introducing the genetic construct of claim 1 into a non-human zygote or an non-human embryonic stem cell,
b) generating a transgenic non-human animal from said zygote or embryonic stem cell, and thereby,
c) producing a transgenic non-human animal whose genome comprises transgenic DNA encoding TAAR1.

3. A method of producing a non-human transgenic animal expressing transgenic TAAR1 comprising
a) introducing the genetic construct of claim 1 into a non-human zygote or an non-human embryonic stem cell,
b) generating a transgenic non-human animal from said zygote or embryonic stem cell, and thereby,
c) producing a transgenic non-human animal expressing transgenic TAAR1.

4. The transgenic non-human animal produced by the method of claim 2.

5. A transgenic non-human animal, whose genome comprises the genetic construct of claim 1.

6. The transgenic non-human animal of claim 4 wherein the transgenic non-human animal is a rodent.

7. The transgenic non-human animal of claim 6 wherein the non-human transgenic animal is a mouse.

8. The transgenic non-human animal according to claim 4 wherein the non-human transgenic animal is C57BL/6.

9. The transgenic non-human animal of claim 4 overexpressing TAAR1 protein.

10. The transgenic non-human animal according to any one of the claim 9 overexpressing TAAR1 protein in a tissue-specific manner.

11. Descendants of the transgenic non-human animal of claim 4.

12. A culture derived from the transgenic non-human animal of claim 4, or its descendants, wherein said culture is selected from the group consisting of a cell line or primary cell culture, a tissue or organotype brain slide culture, a tissue or cell extract, and a tissue or organ explant and cultures thereof.


SEQUENCE LISTING (35 sequences)

SEQ ID NO: 1 - DNA, 1020 bp, Homo sapiens
atgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaacaacttcataccccaacaaattggctcattcattccatggccactgtggactttcttctggggtgtctggtcatgccttacagtatggtgagatctgctgagcactgttggtattttggagaagtcttctgtaaaattcacacaagcaccgacattatgctgagctcagcctccattttccatttgtctttcatctccattgaccgctactatgctgtgtgtgatccactgagatataaagccaagatgaatatcttggttatttgtgtgatgatcttcattagttggagtgtccctgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgttatgtgtctattacagaatatatcttatcgctaaagaacaggcaagattaattagtgatgccaatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatag

SEQ ID NO: 2 - DNA, 999 bp, Mus musculus
atgcatctttgccacgctatcacaaacatttcccacagaaacagcgactggtcaagagaagtccaagcttccctgtacagcttaatgtcactcataatcctggccactctggttggcaacttaatagtaataatttccatatcccatttcaagcaacttcatacacccaccaactggctccttcactccatggccattgtcgactttctgctgggctgtctgataatgccctgcagcatggtgagaactgttgagcgctgttggtattttggggaaatcctctgtaaagttcacaccagcaccgatatcatgctgagctccgcctccattttccacttagctttcatttccattgaccgctactgtgctgtgtgtgaccctttgagatacaaagccaagatcaatatctccactattcttgtgatgatcctcgttagttggagccttcctgctgtttatgcatttgggatgatcttcctggaactgaacttaaaaggagtggaagagctgtatcgcagtcaggtcagcgacctgggcggctgttctcccttctttagtaaagtatctggggtactggcgttcatgacttccttctatatacctggatctgttatgttatttgtttactataggatatatttcatagctaaaggacaagcaaggtcaatcaatcgtacgaatgttcaagttggattggaagggaaaagccaagcaccacaaagcaaggaaacaaaagccgcgaagaccttagggatcatggtgggcgttttcctcgtatgctggtgcccgttctttctctgcacggtcctggaccctttcctgggctatgttatcccaccctctctgaatgacgcactgtattggtttgggtacttgaattctgccctcaatccgatggtttatgcctttttctatccctggttcagaagagccttgaagatggttctccttggtaaaattttccaaaaagattcatctaggtctaagctatttttgtaa

SEQ ID NO: 3 - DNA, 1116 bp, Artificial Sequence
  Description of Artificial Sequence Synthetic construct
ctcgagtccaccatgaagacgatcatcgccctgagctacatcttctgcctggtgttcgccgactacaaggacgatgatgacgccatgggcatgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaacaacttcataccccaacaaattggctcattcattccatggccactgtggactttcttctggggtgtctggtcatgccttacagtatggtgagatctgctgagcactgttggtattttggagaagtcttctgtaaaattcacacaagcaccgacattatgctgagctcagcctccattttccatttgtctttcatctccattgaccgctactatgctgtgtgtgatccactgagatataaagccaagatgaatatcttggttatttgtgtgatgatcttcattagttggagtgtccctgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgttatgtgtctattacagaatatatcttatcgctaaagaacaggcaagattaattagtgatgccaatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatagctcgag

SEQ ID NO: 4 - DNA, 1095 bp, Artificial Sequence
  Description of Artificial Sequence Synthetic construct
ctcgagtccaccatgaagacgatcatcgccctgagctacatcttctgcctggtgttcgccgactacaaggacgatgatgacgccatgggcatgcatctttgccacgctatcacaaacatttcccacagaaacagcgactggtcaagagaagtccaagcttccctgtacagcttaatgtcactcataatcctggccactctggttggcaacttaatagtaataatttccatatcccatttcaagcaacttcatacacccaccaactggctccttcactccatggccattgtcgactttctgctgggctgtctgataatgccctgcagcatggtgagaactgttgagcgctgttggtattttggggaaatcctctgtaaagttcacaccagcaccgatatcatgctgagctccgcctccattttccacttagctttcatttccattgaccgctactgtgctgtgtgtgaccctttgagatacaaagccaagatcaatatctccactattcttgtgatgatcctcgttagttggagccttcctgctgtttatgcatttgggatgatcttcctggaactgaacttaaaaggagtggaagagctgtatcgcagtcaggtcagcgacctgggcggctgttctcccttctttagtaaagtatctggggtactggcgttcatgacttccttctatatacctggatctgttatgttatttgtttactataggatatatttcatagctaaaggacaagcaaggtcaatcaatcgtacgaatgttcaagttggattggaagggaaaagccaagcaccacaaagcaaggaaacaaaagccgcgaagaccttagggatcatggtgggcgttttcctcgtatgctggtgcccgttctttctctgcacggtcctggaccctttcctgggctatgttatcccaccctctctgaatgacgcactgtattggtttgggtacttgaattctgccctcaatccgatggtttatgcctttttctatccctggttcagaagagccttgaagatggttctccttggtaaaattttccaaaaagattcatctaggtctaagctatttttgtaactcgag

SEQ ID NO: 5 - DNA, 410 bp, Artificial Sequence
  Description of Artificial Sequence Synthetic construct
ctcgagtccaccatgaagacgatcatcgccctgagctacatcttctgcctggtgttcgccgactacaaggacgatgatgacgccatgggcatgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaacaacttcataccccaacaaattggctcattcattccatggccactgtggactttcttctggggtgtctggtcatgccttacagtatggtgagatctgctgagcactgttggtattttggagaagtcttctgtaaaattcacacaagcaccgacattatgctgag

SEQ ID NO: 6 - DNA, 345 bp, Artificial Sequence
  Description of Artificial Sequence Synthetic construct
aatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatagctcgag

SEQ ID NO: 7 - DNA, 490 bp, Artificial Sequence
  Description of Artificial Sequence Synthetic construct
ctgttggtattttggagaagtcttctgtaaaattcacacaagcaccgacattatgctgagctcagcctccattttccatttgtctttcatctccattgaccgctactatgctgtgtgtgatccactgagatataaagccaagatgaatatcttggttatttgtgtgatgatcttcattagttggagtgtccctgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgttatgtgtctattacagaatatatcttatcgctaaagaacaggcaagattaattagtgatgccaatcagaagctccaaattggattggaaatgaaaaatggaatttcacaaagcaaagaaaggaaagctgtg

SEQ ID NO: 8 - DNA, 358 bp, Artificial Sequence
  Description of Artificial Sequence Synthetic construct
ctcgagtccaccatgaagacgatcatcgccctgagctacatcttctgcctggtgttcgccgactacaaggacgatgatgacgccatgggcatgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaacaacttcataccccaacaaattggctcattcattccatggccactgtggactttcttctggggtgtctggtcatgccttacagtatggtgagatctgctgagcactgttggt

SEQ ID NO: 9 - DNA, 370 bp, Artificial Sequence
  Description of Artificial Sequence Synthetic construct
acaggcaagattaattagtgatgccaatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatagctcgag

SEQ ID NO: 10 - DNA, 386 bp, Artificial Sequence
  Description of Artificial Sequence Synthetic construct
acagtatggtgagatctgctgagcactgttggtattttggagaagtcttctgtaaaattcacacaagcaccgacattatgctgagctcagcctccattttccatttgtctttcatctccattgaccgctactatgctgtgtgtgatccactgagatataaagccaagatgaatatcttggttatttgtgtgatgatcttcattagttggagtgtccctgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgtta

SEQ ID NO: 11 - DNA, 411 bp, Artificial Sequence
  Description of Artificial Sequence Synthetic construct
ctcgagtccaccatgaagacgatcatcgccctgagctacatcttctgcctggtgttcgccgactacaaggacgatgatgacgccatgggcatgcatctttgccacgctatcacaaacatttcccacagaaacagcgactggtcaagagaagtccaagcttccctgtacagcttaatgtcactcataatcctggccactctggttggcaacttaatagtaataatttccatatcccatttcaagcaacttcatacacccaccaactggctccttcactccatggccattgtcgactttctgctgggctgtctgataatgccctgcagcatggtgagaactgttgagcgctgttggtattttggggaaatcctctgtaaagttcacaccagcaccgatatcatgctgagctcc

SEQ ID NO: 12 - DNA, 378 bp, Artificial Sequence
  Description of Artificial Sequence Synthetic construct
tataggatatatttcatagctaaaggacaagcaaggtcaatcaatcgtacgaatgttcaagttggattggaagggaaaagccaagcaccacaaagcaaggaaacaaaagccgcgaagaccttagggatcatggtgggcgttttcctcgtatgctggtgcccgttctttctctgcacggtcctggaccctttcctgggctatgttatcccaccctctctgaatgacgcactgtattggtttgggtacttgaattctgccctcaatccgatggtttatgcctttttctatccctggttcagaagagccttgaagatggttctccttggtaaaattttccaaaaagattcatctaggtctaagctatttttgtaactcgag

SEQ ID NO: 13 - DNA, 371 bp, Artificial Sequence
  Description of Artificial Sequence Synthetic construct
gtgagaactgttgagcgctgttggtattttggggaaatcctctgtaaagttcacaccagcaccgatatcatgctgagctccgcctccattttccacttagctttcatttccattgaccgctactgtgctgtgtgtgaccctttgagatacaaagccaagatcaatatctccactattcttgtgatgatcctcgttagttggagccttcctgctgtttatgcatttgggatgatcttcctggaactgaacttaaaaggagtggaagagctgtatcgcagtcaggtcagcgacctgggcggctgttctcccttctttagtaaagtatctggggtactggcgttcatgacttccttctatatacctggatctgt

SEQ ID NO: 14 - DNA, 292 bp, Artificial Sequence
  Description of Artificial Sequence Synthetic construct
ctcgagtccaccatgaagacgatcatcgccctgagctacatcttctgcctggtgttcgccgactacaaggacgatgatgacgccatgggcatgcatctttgccacgctatcacaaacatttcccacagaaacagcgactggtcaagagaagtccaagcttccctgtacagcttaatgtcactcataatcctggccactctggttggcaacttaatagtaataatttccatatcccatttcaagcaacttcatacacccaccaactggctccttcactccatggccattgtcg

SEQ ID NO: 15 - DNA, 391 bp, Artificial Sequence
  Description of Artificial Sequence Synthetic construct
gttatttgtttactataggatatatttcatagctaaaggacaagcaaggtcaatcaatcgtacgaatgttcaagttggattggaagggaaaagccaagcaccacaaagcaaggaaacaaaagccgcgaagaccttagggatcatggtgggcgttttcctcgtatgctggtgcccgttctttctctgcacggtcctggaccctttcctgggctatgttatcccaccctctctgaatgacgcactgtattggtttgggtacttgaattctgccctcaatccgatggtttatgcctttttctatccctggttcagaagagccttgaagatggttctccttggtaaaattttccaaaaagattcatctaggtctaagctatttttgtaactcgag

SEQ ID NO: 16 - DNA, 451 bp, Artificial Sequence
  Description of Artificial Sequence Synthetic construct
cccaccaactggctccttcactccatggccattgtcgactttctgctgggctgtctgataatgccctgcagcatggtgagaactgttgagcgctgttggtattttggggaaatcctctgtaaagttcacaccagcaccgatatcatgctgagctccgcctccattttccacttagctttcatttccattgaccgctactgtgctgtgtgtgaccctttgagatacaaagccaagatcaatatctccactattcttgtgatgatcctcgttagttggagccttcctgctgtttatgcatttgggatgatcttcctggaactgaacttaaaaggagtggaagagctgtatcgcagtcaggtcagcgacctgggcggctgttctcccttctttagtaaagtatctggggtactggcgttcatgacttccttctatatacctggatctgttatgt

SEQ ID NO: 17 - DNA, 21 bp, Artificial Sequence
  Description of Artificial Sequence Synthetic oligonucleotide
cttctggggtgtctggtcatg

SEQ ID NO: 18 - DNA, 22 bp, Artificial Sequence
  Description of Artificial Sequence Synthetic oligonucleotide
gcataatgtcggtgcttgtgtg

SEQ ID NO: 19 - DNA, 24 bp, Artificial Sequence
  Description of Artificial Sequence Synthetic oligonucleotide
cctggatctattatgttatgtgtc

SEQ ID NO: 20 - DNA, 23 bp, Artificial Sequence
  Description of Artificial Sequence Synthetic oligonucleotide
cttgcctgttctttagcgataag

SEQ ID NO: 21 - DNA, 112 bp, Artificial Sequence
  Description of Artificial Sequence Synthetic oligonucleotide
gcctcgagtccaccatgaagacgatcatcgccctgagctacatcttctgcctggtgttcgccgactacaaggacgatgatgacgccatgggcatgatgcccttttgccacaa

SEQ ID NO: 22 - DNA, 28 bp, Artificial Sequence
  Description of Artificial Sequence Synthetic oligonucleotide
gcctcgagctatgaactcaattccaaaa

SEQ ID NO: 23 - DNA, 20 bp, Artificial Sequence
  Description of Artificial Sequence Synthetic oligonucleotide
cgactttctgctgggctgtc

SEQ ID NO: 24 - DNA, 21 bp, Artificial Sequence
  Description of Artificial Sequence Synthetic oligonucleotide
caacagttctcaccatgctgc

SEQ ID NO: 25 - DNA, 21 bp, Artificial Sequence
  Description of Artificial Sequence Synthetic oligonucleotide
gtactggcgttcatgacttcc

SEQ ID NO: 26 - DNA, 22 bp, Artificial Sequence
  Description of Artificial Sequence Synthetic oligonucleotide
ccttgcttgtcctttagctatg

SEQ ID NO: 27 - DNA, 115 bp, Artificial Sequence
  Description of Artificial Sequence Synthetic oligonucleotide
gcctcgagtccaccatgaagacgatcatcgccctgagctacatcttctgcctggtgttcgccgactacaaggacgatgatgacgccatgggcatgcatctttgccacgctatcac

SEQ ID NO: 28 - DNA, 28 bp, Artificial Sequence
  Description of Artificial Sequence Synthetic oligonucleotide
gcctcgagttacaaaaatagcttagacc

SEQ ID NO: 29 - DNA, 20 bp, Artificial Sequence
  Description of Artificial Sequence Synthetic oligonucleotide
aattaaccctcactaaaggg

SEQ ID NO: 30 - DNA, 20 bp, Artificial Sequence
  Description of Artificial Sequence Synthetic oligonucleotide
taatacgactcactataggg

SEQ ID NO: 31 - DNA, 20 bp, Artificial Sequence
  Description of Artificial Sequence Synthetic oligonucleotide
gtgctccgtggatctcaagc

SEQ ID NO: 32 - DNA, 21 bp, Artificial Sequence
  Description of Artificial Sequence Synthetic oligonucleotide
ggatgatggcatgcagcactg

SEQ ID NO: 33 - DNA, 18 bp, Artificial Sequence
  Description of Artificial Sequence Synthetic primer
gatcatcgccctgagcta

SEQ ID NO: 34 - DNA, 20 bp, Artificial Sequence
  Description of Artificial Sequence Synthetic primer
gcgtcatcatcgtccttgta

SEQ ID NO: 35 - DNA, 16 bp, Artificial Sequence
  Description of Artificial Sequence Synthetic probe
tcttctgcctggtgtt